(19) United States
(12) Patent Application Publication
(10) Pub. No.: US 2026/0108715 A1
(43) Pub. Date: Apr. 23, 2026

(54) COLLAGEN STIMULATION DEVICE

(71) Applicant: CROWN LABORATORIES, INC., Johnson City, TN (US)

(72) Inventor: Thomas M. HITCHCOCK, Dallas, TX (US)

(21) Appl. No.: 19/364,925

(22) Filed: Oct. 21, 2025

Related U.S. Application Data
(60) Provisional application No. 63/709,952, filed on Oct. 21, 2024.

(51) Int. Cl. A61M 37/00; A61B 90/90

(52) U.S. Cl. CPC A61M 37/0015; A61B 90/90; A61M 2037/0061; A61M 2205/273; A61M 2205/502; A61M 2210/04

(57) ABSTRACT

A collagen stimulation assembly includes a pen and a single user cartridge. The pen includes a motor, a first attachment portion configured to be rotated about an axis, and a transmitter. The single use cartridge includes a second attachment portion coupled to the first attachment portion, a reciprocation assembly, a plurality of needles connected to the reciprocation assembly, and an RFID tag configured to interact with the transmitter. The plurality of needles is configured to move along the axis. The transmitter detects a status of the RFID tag in response to the second attachment portion coupling to the first attachment portion. The motor can operate when the status is a first value. The motor is prevented from operating when the status is a second value. The transmitter changes the status to the second value in response to the second attachment portion detaching from the first attachment portion.

### CROSS-REFERENCE TO RELATED APPLICATIONS

This application claims priority to U.S. Provisional Patent Application No. 63/709,952, which was filed on Oct. 21, 2024, and entitled “Collagen Stimulation Device,” the entire contents of which is hereby incorporated by reference in its entirety.

### FIELD

The present disclosure relates to a device that utilizes needles to generate a controlled micro-injury to stimulate collagen growth in the epidermis. More specifically, the present disclosure incorporates an electronic lockout system into a collagen stimulation assembly to ensure a new set of needles is used during each operation.

### BACKGROUND

Collagen growth is stimulated by injury to the skin. Uncontrolled injury of the dermis can trigger collagen over stimulation and create scar tissue and keloids. Controlled micro-injury to the dermis causes collagen to be produced in a relatively controlled manner to restore elasticity to older skin. Additionally, in the cases of scar tissue and keloids, micro-injury can cause remodeling of the tissue to reduce the overall appearance of the scar.

### SUMMARY

In one example of an embodiment, a collagen stimulation assembly includes a pen and a single user cartridge. The pen includes a motor, a first attachment portion configured to be rotated by the motor about an axis, and a transmitter. The single use cartridge includes a second attachment portion coupled to the first attachment portion, a reciprocation assembly connected to the second attachment portion, a plurality of needles connected to the reciprocation assembly, and an RFID tag configured to interact with the transmitter. The second attachment portion is configured to be rotated about the axis by the first attachment portion. The reciprocation assembly is configured to move along the axis. The plurality of needles is configured to move along the axis with the reciprocation assembly. The transmitter detects a status of the RFID tag in response to the second attachment portion coupling to the first attachment portion. The motor can operate when the status is a first value. The motor is prevented from operating when the status is a second value. The transmitter changes the status to the second value in response to the second attachment portion detaching from the first attachment portion.

In another example of an embodiment, a collagen stimulation assembly includes a pen and a single use cartridge. The pen includes a motor, a first star shaped attachment portion configured to be rotated by the motor about an axis, and a transmitter. The single use cartridge includes a second star shaped attachment portion coupled to the first star shaped attachment portion, a reciprocation assembly connected to the second star shaped attachment portion, a plurality of needles connected to the reciprocation assembly, and a wireless tag configured to interact with the transmitter. The second star shaped attachment portion is configured to be rotated about the axis by the first star shaped attachment portion. The reciprocation assembly is configured to move along the axis. The plurality of needles is configured to move along the axis with the reciprocation assembly. The transmitter detects a status of the wireless tag in response to the second attachment portion coupling to the first attachment portion. The motor can operate when the status is a first value. The motor is prevented from operating when the status is a second value. The transmitter changes the status to the second value in response to the second attachment portion detaching from the first attachment portion.

In yet another example of an embodiment, a method of operating a collagen stimulation assembly includes a pen and a single use cartridge. The pen has a motor, a first attachment portion configured to be rotated by the motor about an axis, and a transmitter. The single use cartridge has a second attachment portion, a reciprocation assembly connected to the second attachment portion, a plurality of needles connected to the reciprocation assembly, and a wireless tag. The method includes connecting the second attachment portion to the first connection portion, detecting a status of the wireless tag with the transmitter, the status being a first value or a second value, preventing operation of the motor in response to the status being the second value, operating the motor in response to the status being the first value, operating the motor causing the first attachment portion and the second attachment portion to rotate about the axis, rotation of the second attachment portion causing the reciprocation assembly and the plurality of needles to reciprocate along the axis, ending operation of the motor, detaching the second attachment portion from the first connection portion, and adjusting the status of the wireless tag to the second value with the transmitter.

Other aspects of the invention will become apparent by consideration of the detailed description and accompanying drawings.

### BRIEF DESCRIPTION OF THE DRAWINGS

FIG. 1 is a profile view of an example of an embodiment of a collagen stimulation assembly including a pen and a cartridge.

FIG. 2 is a perspective view of the pen of the collagen stimulation assembly shown in FIG. 1.

FIG. 3 is an exploded view of the pen of FIG. 2.

FIG. 4 is a perspective view of the cartridge of the collagen stimulation assembly shown in FIG. 1 illustrating a first end.

FIG. 5 is a perspective view of the cartridge of FIG. 4 illustrating a second end opposite the first end.

FIG. 6 is an exploded view of the cartridge of FIG. 4.

FIG. 7 is a cross-sectional view of the cartridge taken along line 7-7 in FIG. 4.

FIG. 8 is a cross-sectional view of the cartridge taken along line 8-8 in FIG. 4.

FIG. 9 is a partially exploded view of an alternative example of an embodiment of the cartridge of FIG. 4.

FIG. 10 is a partially exploded view of the cartridge of FIG. 9 illustrating a cup disengaged from a second reciprocation rod.

FIG. 11 is a side view of the cartridge of FIG. 10 illustrating the cup disengaged from the second reciprocation rod.

FIG. 12 is a perspective view of the cup and second reciprocation rod of FIG. 11 illustrating a fastener assembly.

FIG. 13 is a perspective view of a charger for use with the pen of FIG. 2.

FIG. 14 is a flow diagram providing an example of an embodiment of a process for operating the collagen stimulation assembly of FIG. 1.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

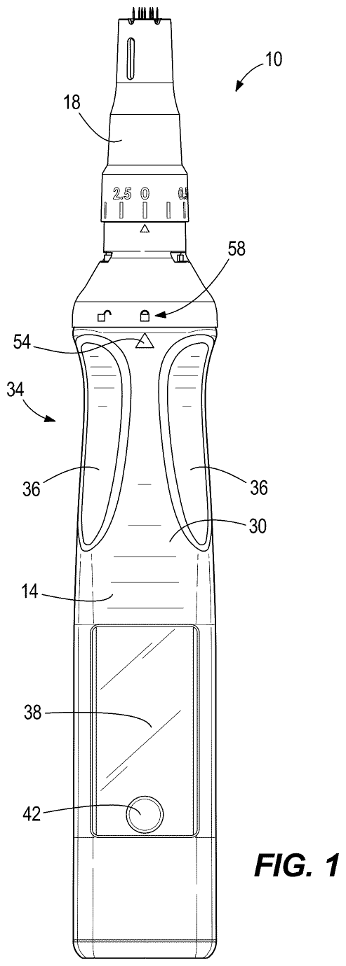

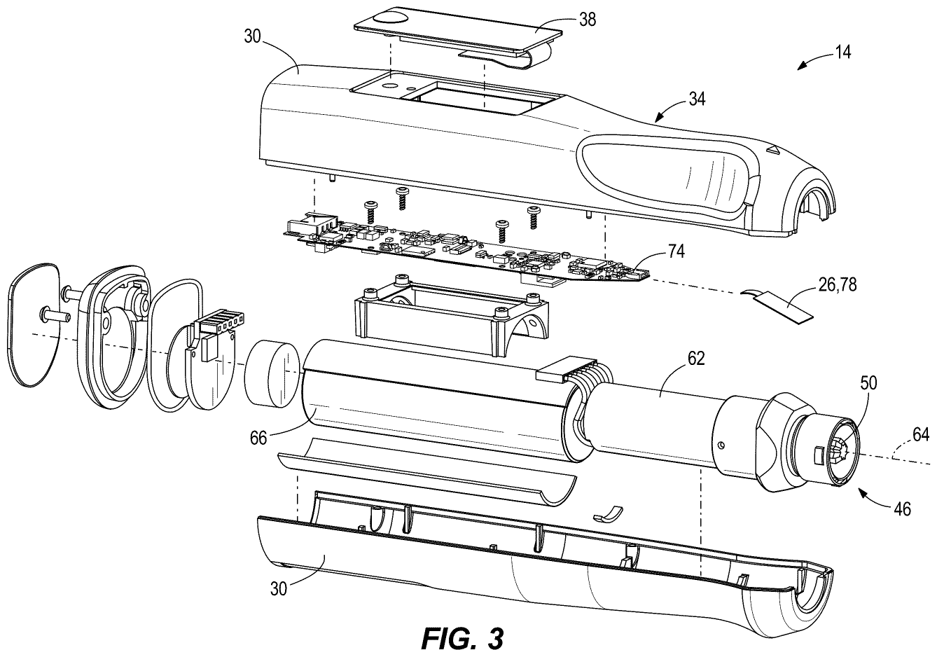

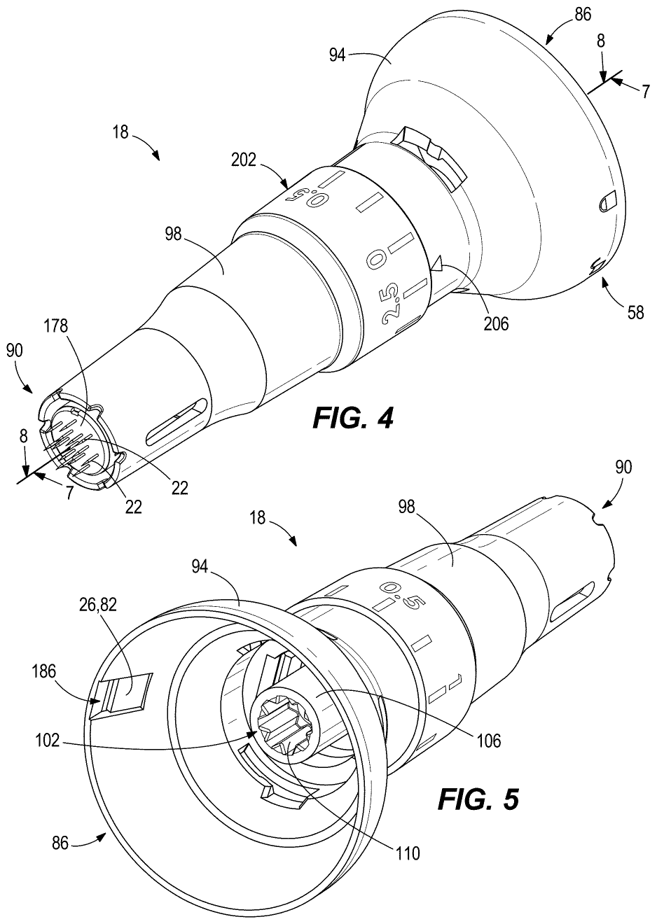

FIG. 1 illustrates an example of an embodiment of a collagen stimulation assembly 10 configured to micro-injure skin. The collagen stimulation assembly 10 including a pen 14 and a cartridge 18. The cartridge 18 (also referred to as a single use cartridge 18) is removably coupled to the pen 14. The pen 14 generates a rotational motion, which is converted to a reciprocation motion by the cartridge 18. The reciprocation motion is transferred to a plurality of needles 22 (shown in FIG. 4) positioned within the cartridge 18. The reciprocating needles 22 are configured to micro-injure skin (e.g., human skin) in a controlled manner to stimulate collagen growth in the skin. The cartridge 18 is one of a plurality of cartridges 18 that can be coupled to the pen 14. The collagen stimulation assembly 10 includes an electronic lockout system 26 (shown in FIG. 3) to prevent one cartridge 18 from being used more than once. The electronic lockout system 26 can, for example, prevent the pen 14 from operating when a used cartridge 18 is attached to the pen 14.

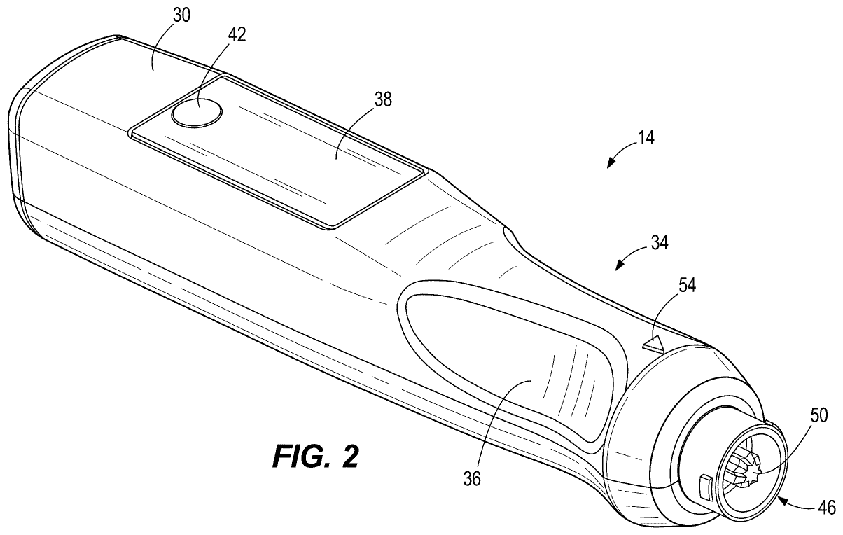

With reference to FIGS. 1 and 2, the pen 14 includes a pen housing 30. The illustrated pen housing 30 includes a grip portion 34. The grip portion 34 can include recessed portions 36, which improve an operator's ability to grasp the pen 14 and control movement of the pen 14. The pen 14 includes a display 38. The display 38 is coupled to an outer side of the housing 30. The display 38 can be a digital display configured to provide information, such as performance, status, and/or any suitable operational feature of the collagen stimulation assembly 10, which is described in further detail below. The pen 14 also includes a button 42. The button 42 is positioned adjacent the display 38. The button 42 is connected to the display 38 and allows an operator to interact with the display 38. The button 42 can include a primary function. In some examples of embodiments, the primary function can be to stop or start reciprocation of the needles 22. The button 42 can further include at least one secondary function. In some examples of embodiments, the secondary function can be to power on and off the pen 14. The primary function and the secondary function can be initiated by different actuations of the button 42. In some examples of embodiments, the primary function can be initiated by a press of the button 42 for a first period of time (e.g., less than one second, one half second, etc.), and the secondary function can be initiated by a press of the button 42 for a second period of time different than the first period of time. For example, the secondary function can be initiated by a press of the button 42 for the second period of time (e.g., two seconds, three seconds, etc.), which is longer than the first period of time. In other examples of embodiments, the primary function can be initiated by a first actuation of the button 42 (e.g., a press of the button 42) while the secondary function can be initiated by a second actuation of the button 42 (e.g., rotation of the button 42). In this example, the movement of the button 42 differs to initiate the respective function.

With reference to FIG. 2, the pen 14 includes an attachment portion 46 (also referred to as a first attachment portion 46). The attachment portion 46 connects the pen 14 to the cartridge 18 (shown in FIG. 4). The attachment portion 46 includes an attachment shaft 50 (also referred to as a drive shaft 50 or shaft 50). The illustrated attachment shaft 50 defines an end having an interlocking geometry configured to selectively engage an attachment portion 102 of the cartridge 18 (shown in FIG. 5). In the illustrated embodiment, the attachment shaft 50 is star shaped. In other examples of embodiments, the attachment shaft 50 can have a different shape that is suitable to interlock with the attachment portion 102 of the cartridge 18.

With continued reference to FIG. 2, the pen 14 includes a locking indicia assembly that includes a first indicia 54 on the outer side of the housing 30. The first indicia 54 is configured to align with a second indicia 58 (shown in FIG. 1) on the cartridge 18 to indicate if the cartridge 18 is fastened (or unfastened) to the pen 14. The second indicia 58 can include a plurality of visual representations configured to visually communicate the locking configuration of the cartridge 18 relative to the housing 30. The illustrated second indicia 58 include a first symbol (represented by a lock symbol) and a second symbol (represented by an unlocked symbol). When the first indicia 54 is aligned with the lock symbol, the cartridge 18 is locked to the pen 14. The collagen stimulation assembly 10 can be operated when the cartridge 18 is locked to the pen 14. When the first indicia 54 is aligned with the unlocked symbol, the cartridge 18 is unlocked from the pen 14. The cartridge 18 can be removed from the pen 14 when the cartridge 18 is unlocked from the pen 14. The cartridge 18 and/or pen 14 may output tactile feedback to further indicate that the cartridge 18 is either locked or unlocked to the pen 14. The tactile feedback can be a vibration, an audible click, or any suitable feedback to indicate that the cartridge 18 is either locked or unlocked to the pen 14.

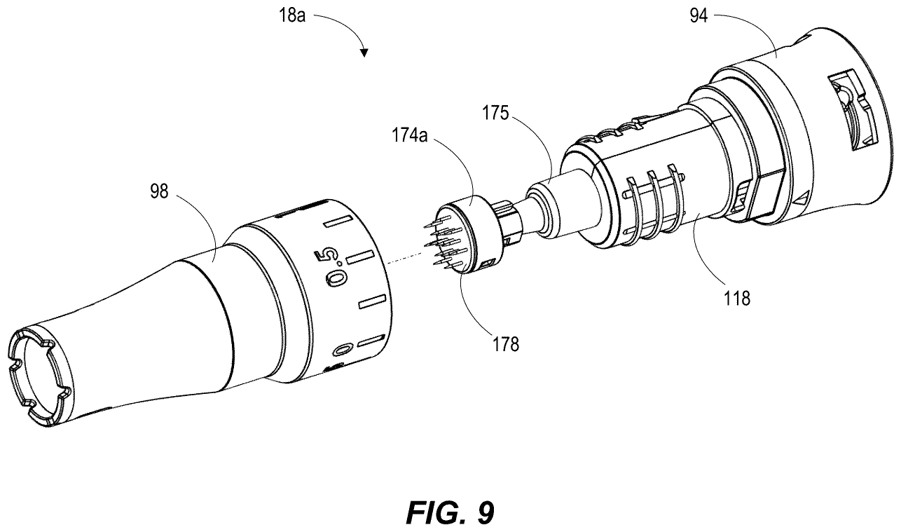

With reference now to FIG. 3, the pen 14 includes a motor 62. The motor 62 is received by the housing 30 and operably connected to the attachment shaft 50. The motor 62 is configured to rotate the attachment shaft 50 about an axis 64 (also referred to as a rotational axis 64). The motor 62 is powered by a battery 66 that is electrically connected to the motor 62. The battery 66 is configured to be recharged by a charger 70 (shown in FIG. 9). The pen 14 also includes a controller 74. The controller 74 can be, for example, a printed circuit board (PCB). The controller 74 is connected to the display 38, the motor 62, and the battery 66. The controller 74 can receive signals from the motor 62 (e.g., torque, rotations per unit time, rotational speed, etc.) and/or the battery 66 (e.g., battery life, battery charge status, etc.) and output the data on the display 38 for an operator to view.

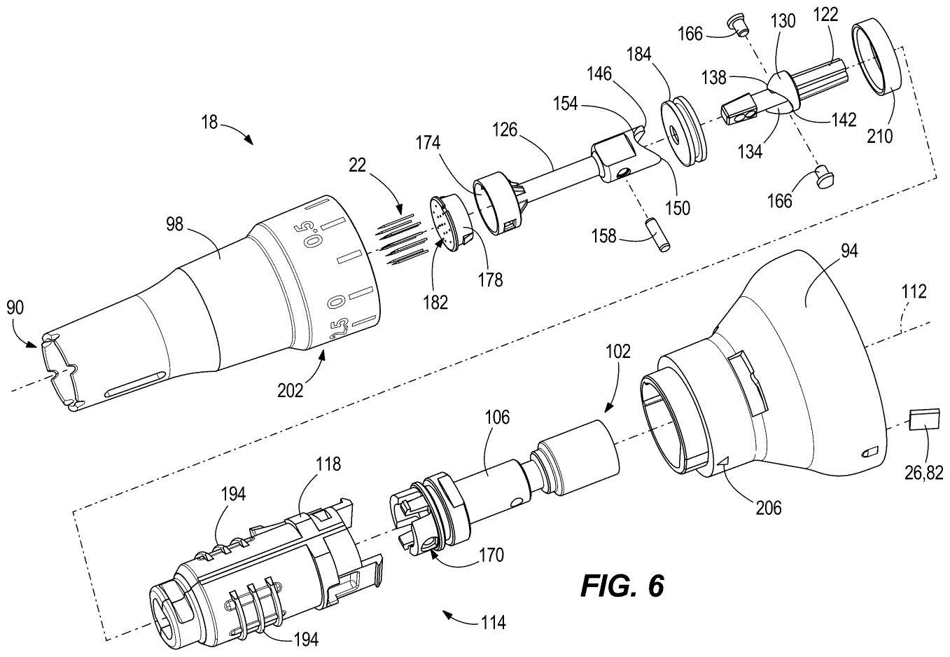

With continued reference to FIG. 3, the pen 14 includes a transmitter 78. The transmitter 78 is operably connected to the controller 74 and the display 38. The transmitter 78 is configured to communicate with a wireless tag 82 (shown in FIG. 6) that is positioned within the cartridge 18. The communication between the transmitter 78 and wireless tag 82 is to prevent a cartridge 18 from being used more than once. The transmitter 78 and the wireless tag 82 are components of the lockout system 26, which is described in further detail below.

With reference to FIGS. 4 and 5, the cartridge 18 includes an attachment end 86 (also referred to as a first end 86) and an operational end 90 (also referred to as a second end 90). The operational end 90 is positioned opposite the attachment end 86. The attachment end 86 is configured to attach to or engage the pen 14. The needles 22 are located at the operational end 90. The cartridge 18 includes a cartridge housing assembly 92. The cartridge housing assembly 92 can include a first cartridge housing 94 and a second cartridge housing 98. The first cartridge housing 94 defines the attachment end 86, and the second cartridge housing 98 defines the operational end 90. In other examples of embodiments, the cartridge housing assembly 92 can include a single unitary housing 94 or 98. In yet other examples of embodiments, the first cartridge housing 94 can be a first cartridge housing portion, and the second cartridge housing 98 can be a second cartridge housing portion, where the portions define respective components or areas of the cartridge housing assembly 92.

With specific reference to FIG. 5, the cartridge 18 includes the attachment portion 102 (also referred to as a second attachment portion 102) on an intermediate drive 106. The attachment portion 102 includes a receiving bore 110. The receiving bore 110 receives the attachment shaft 50 (shown in FIG. 3) to couple (or operably connect) the cartridge 18 to the pen 14. Rotation of the attachment shaft 50 by the motor 62 responsively causes the intermediate drive 106 to rotate about an axis 112 (shown in FIG. 6). The axis 112 and as the axis 64 (shown in FIG. 3) are parallel and overlapping.

The illustrated receiving bore 110 has a shape complimentary to the attachment shaft 50 (shown in FIG. 3). More specifically, the receiving bore 110 defines a star shaped socket. The star shaped attachment shaft 50 and receiving bore 110 provides an improved connection between the motor 62 and the intermediate drive 106. The plurality of points (e.g., six, eight, ten, etc.) on the star shape increases the engagement surface between the attachment shaft 50 and the receiving bore 110. The larger engagement surface increases the amount of contact between the attachment shaft 50 and the receiving bore 110. The increased contact improves the rotation of the intermediate drive 106 with the attachment shaft 50. Stated another way, the increased contact between the attachment shaft 50 and the receiving bore 110 allows for an improved transfer of torque from the attachment shaft 50 to the intermediate drive 106 while limiting a risk of slipping (or camming out) that can damage the attachment shaft 50 and/or the receiving bore 110.

With reference now to FIG. 6, the cartridge 18 includes a reciprocation assembly 114. The reciprocation assembly 114 includes the intermediate drive 106, an intermediate housing 118, a first reciprocation rod 122, and a second reciprocation rod 126. The intermediate drive 106, the first reciprocation rod 122, and the second reciprocation rod 126 are each positioned at least partially within the intermediate housing 118. The first reciprocation rod 122 includes a radial flange 130. The radial flange 130 defines a first surface 134 (also referred to as a first cam surface 134). The first surface 134 includes a first raised portion 138 and a first depressed portion 142. The illustrated first surface 134 includes two first raised portions 138 and two first depressed portions 142 in an alternating fashion.

With continued reference to FIG. 6, the second reciprocation rod 126 includes a second surface 146 (also referred to as a second cam surface 146). The second surface 146 includes a second raised portion 150 and a second depressed portion 154. The illustrated second surface 146 includes two second raised portions 150 and two second depressed portions 154 in an alternating fashion.

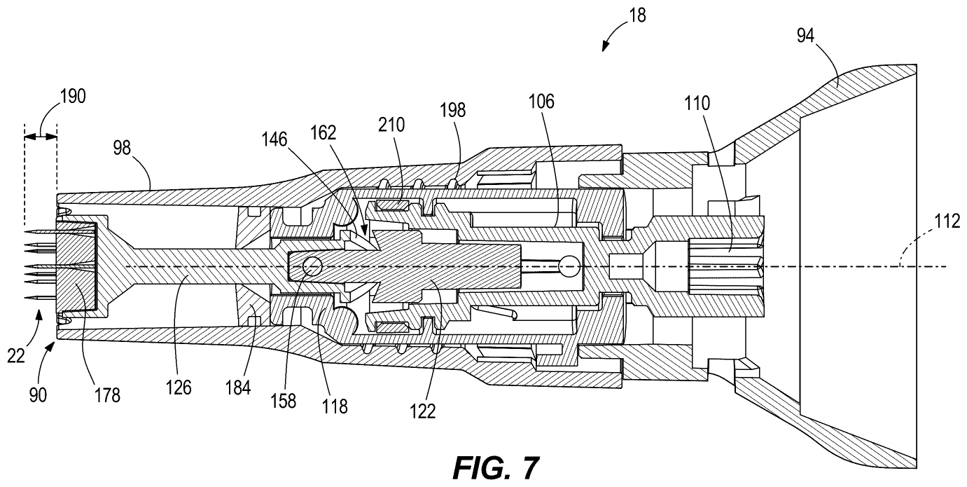

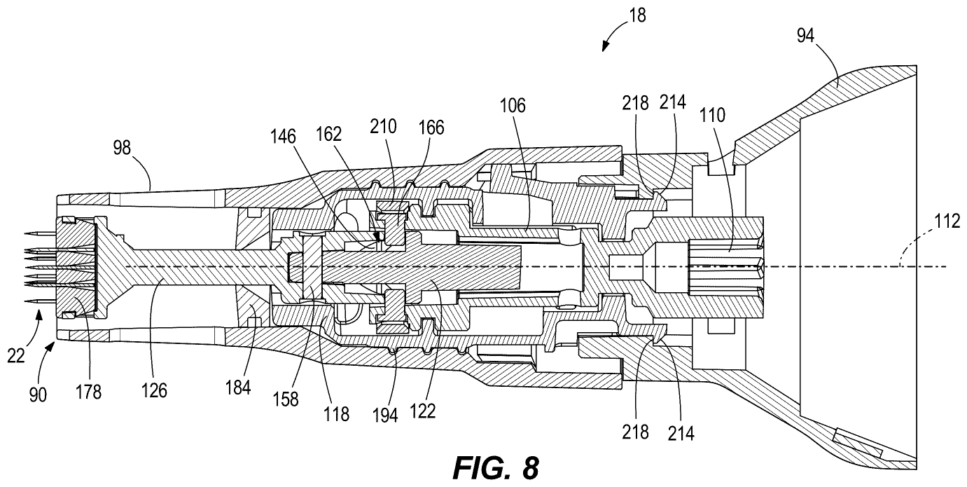

The first and second reciprocation rods 122, 126 are coupled together by a pin 158 extending through the first and second reciprocation rods 122, 126. The first surface 134 and the second surface 146 are spaced apart from each other and together define a channel 162 (shown in FIGS. 7 and 8). The first and second surfaces 134, 146 are aligned such that each first raised portion 138 is adjacent one second depressed portion 154 and each first depressed portion 142 is adjacent one second raised portion 150. In other examples of embodiments, the reciprocation assembly 114 can include fewer or more first raised portions 138, first depressed portions 142, second raised portions 150, and second depressed portions 154. However, in each embodiment, the number of first raised portions 138 and second depressed portions 154 is equal, and the number of first depressed portions 142 and second raised portions 150 is equal. In the illustrated embodiment, the first raised portions 138, first depressed portions 142, second raised portions 150, and second depressed portions 154 are curved or arcuate.

With continued reference to FIG. 6, a slider 166 (also referred to as a pin 166) is coupled to the intermediate drive 106. More specifically, the slider 166 is received in a slider aperture 170 in the intermediate drive 106. The slider 166 rotates with the intermediate drive 106 about the axis 112. In the illustrated embodiment, the reciprocation assembly 114 includes two sliders 166 received in two slider apertures 170, respectively. In other embodiments, the reciprocation assembly 114 can include fewer or more sliders 166 and slider apertures 170 (e.g., one, three, four, etc.). Each slider 166 is received in the channel 162 (shown in FIG. 8). As the intermediate drive 106 and each slider 166 is rotated about the axis 112, each slider 166 moves along the channel 162. The channel 162 is shaped to guide each slider 166 along a specific track or path. For example, the channel 162 can define a sine wave track, a double sine wave track, a sinusoidal wave track, a double sinusoidal wave track, etc. As the slider 166 moves along the path of the channel 162, the first and second reciprocation rods 122, 126 are reciprocated along the axis 112. The path determines the number of reciprocations of the first and second reciprocation rods 122, 126 per rotation of the intermediate drive 106. For example, in embodiments with the channel 162 defining a sine wave track, one full rotation of the intermediate drive 106 (i.e., 360 degrees) causes the first and second rods 122, 126, and thus the needles 22, to reciprocate once (i.e., move linearly in a first direction away from a starting point and then move linearly in a second direction opposite the first direction to return to the starting point). In embodiments with the channel 162 defining a double sine wave track, one full rotation of the intermediate drive 106 causes the first and second rods 122, 126, and thus the needles 22, to reciprocate twice. In other examples of embodiments, the channel 162 can define any track to achieve a suitable number of reciprocations of the needles 22 per rotation of the intermediate drive 106 (e.g., two and a half, three, five, etc.).

The second reciprocation rod 126 includes a cup 174. The cup 174 is on a side of the second reciprocation rod 126 opposite the second surface 146. A needle holder 178 is received in the cup 174. The needle holder 178 can be coupled to the cup 174 by a snap fit, a press fit, a fastener, etc. The needle holder 178 includes (or defines) a plurality of bores 182. One of the plurality of needles 22 is received in one of the plurality of bores 182. Stated another way, each bore 182 receives a needle 22. The needles 22 reciprocate with the first and second reciprocation rods 122, 126 and the needle holder 178 along the axis 112. As the needles 22 reciprocate, the needles 22 are configured to micro-injure skin (e.g., human skin) in a controlled manner to stimulate collagen growth in the skin. The illustrated cartridge 18 includes fourteen needles 22. In other examples of embodiments, the cartridge 18 can include any suitable number of needles 22 (e.g., ten, twelve, sixteen, etc.) to micro-injure skin in a controlled manner to stimulate collagen growth in the skin.

A washer 184 (also referred to as an alignment washer 184) is positioned on the second reciprocation rod 126 between the second surface 146 and the cup 174. The washer 184 reciprocates with the intermediate drive 106. The washer 184 can engage an inner surface of the second cartridge housing 98 to ensure the second reciprocation rod 126 moves along the axis 112 (shown in FIG. 7). This improves the accuracy of the needles 22.

The cartridge 18 includes the wireless tag 82. The wireless tag 82 can be an RFID tag. The illustrated wireless tag 82 is positioned within a slot 186 (shown in FIG. 5) in the first cartridge housing 94. The wireless tag 82 is configured to include a status. The status can be a first value or a second value. The first value can indicate that the cartridge 18 has not been used, and the second value can indicate that the cartridge 18 has been used. The transmitter 78 is configured to detect the status of the wireless tag 82. For example, the transmitter 78 can be configured to detect the status of the wireless tag 82 in response to the cartridge 18 being coupled to the pen 14. The transmitter 78 can also be configured to change the status of the wireless tag 82. For example, the transmitter 78 can change the status stored on the wireless tag 82 from the first value to the second value in response to removal (or detachment) of the cartridge 18 from the pen 14. The controller 74 can allow operation of the motor 62 when the status is the first value and prevent operation of the motor 62 when the status is the second value. The status can also be shown on the display 38. For example, when the status is the first value, the display 38 can indicate that the collagen stimulation assembly 10 is operational. A user can then, for example, press the button 42 to activate the motor 62 and operate the collagen stimulation assembly 10. When the status is the second value, the display 38 can indicate that the collagen stimulation assembly 10 is not operational. Stated another way, the cartridge 18 cannot be used with the pen 14. As such, if the user presses the button 42, the motor 62 will not be activated to operate the collagen stimulation assembly 10.

In the illustrated embodiment, the wireless tag 82 is positioned adjacent the transmitter 78, such that the wireless tag 82 is sufficiently close to the transmitter 78 to identify and interact with the wireless tag 82. In other examples of embodiments, the transmitter 78 can be positioned elsewhere in the pen 14, and the wireless tag 82 can be positioned elsewhere in the cartridge 18. However, in every embodiment, the transmitter 78 and the wireless tag 82 are positioned to facilitate communication between the transmitter 78 and the wireless tag 82.

With reference now to FIG. 7, the second cartridge housing 98 is rotatable relative to the first cartridge housing 94 to adjust an insertion depth 190 (also referred to as a needle depth 190) of the needles 22. The insertion depth 190 is defined as the distance between the tips of the needles 22 and the operational end 90 (or an end of the second cartridge housing 98). The insertion depth 190 is the depth the needles 22 extend into the skin. The intermediate housing 118 includes first threads 194 (shown in FIG. 6) extending along an external surface of the intermediate housing 118. The second cartridge housing 98 includes second threads 198 extending along an internal surface of the second cartridge housing 98. The first threads 194 engage the second threads 198. The second cartridge housing 98 moves along the axis 112 relative to the rest of the cartridge 18 (e.g., the needles 22, the first cartridge housing 94, the intermediate housing 118, etc.) as the second cartridge housing 98 is rotated relative to the intermediate housing 118 about the first and second threads 194, 198.

With reference back to FIG. 6, the cartridge 18 can include measurement indicia 202 and an associated pointer 206. For example, the second cartridge housing 98 can include the measurement indicia 202, while the first cartridge housing 94 can include the pointer 206. The measurement indicia 202 lists a plurality of numerical values that are associated with the insertion depth 190 of the needles 22. The pointer 206 can indicate which numerical value is selected based on a position of the second cartridge housing 98 relative to the first cartridge housing 94. The measurement indicia 202 can include numerical values in millimeters, centimeters, inches, or any other suitable unit of measurement. In one example of an embodiment, the numerical values can indicate the value of the insertion depth 190. As such, if the pointer 206 is adjacent a number two, the insertion depth 190 can be equal to two millimeters (or other unit of measurement). In another example of an embodiment, the measurement indicia 202 can indicate a numerical value of difference between an original insertion depth and the actual insertion depth 190. For example, if an original insertion depth is equal to three millimeters, and the pointer 206 is adjacent a number one, the insertion depth 190 can be equal to three minus one or two millimeters. The cartridge 18 may output tactile feedback as the second cartridge housing 98 rotates relative to the first cartridge housing 94. The tactile feedback can be a vibration, an audible click, or any suitable feedback to indicate a relative positioning of the measurement indicia 202 and the pointer 206. For example, the cartridge 18 may output tactile feedback every quarter millimeter of adjustment of the insertion depth 190.

With reference to FIG. 8, the cartridge 18 includes a gasket 210 surrounding the intermediate drive 106 and each slider 166 within the intermediate housing 118. The gasket 210 can retain each slider 166 within the respective slider aperture 170. The gasket 210 can allow each slider 166 to not be rigidly coupled (e.g., by fasteners, glue, etc.) to the intermediate drive 106. As such, the gasket 210 can retain each slider 166 in the respective slider aperture 170 while allowing each slider 166 to move radially due to vibrations, impact, etc. This can improve the lifespan of each slider 166.

The intermediate drive 106 includes an engagement portion 214. The engagement portion 214 engages a ledge 218 on the first cartridge housing 94 to couple the intermediate drive 106 to the first cartridge housing 94.

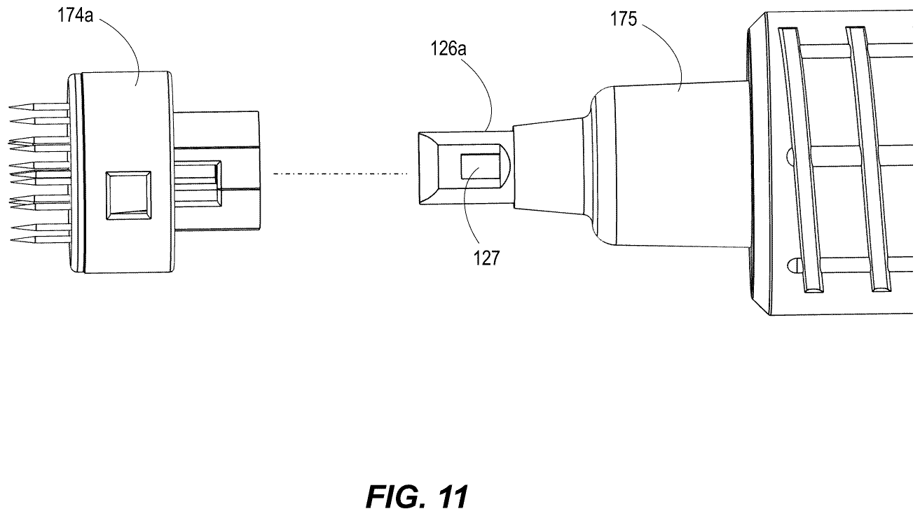

FIGS. 9-12 illustrate an alternative embodiment of a cartridge 18a. It should be appreciated that the cartridge 18a is substantially the same as the cartridge 18, with like numbers identifying like components. With specific reference to FIG. 9, the cartridge 18a is illustrated with the second cartridge housing 98 disengaged from the first cartridge housing 94 to illustrate a removable cup 174a. The cup 174a is configured to receive the needle holder 178. The needle holder 178 can be removably attached (or otherwise removably fastened or removably coupled) to the cup 174a. As a non-limiting example, the needle holder 178 can be removably connected to the cup 174a by a snap fit, a press fit, a fastener, or any other suitable connection.

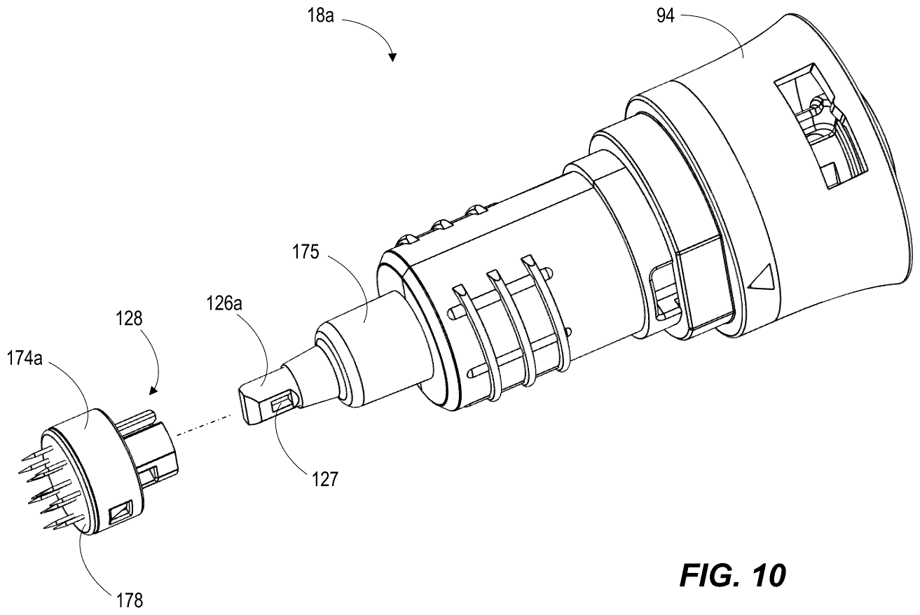

With reference to FIGS. 10-12, the cup 174a is illustrated as detached from a second reciprocation rod 126a. Accordingly, the cup 174a can be removably attached (or otherwise removably fastened or removably coupled) to the second reciprocation rod 126a. In the illustrated embodiment, the second reciprocation rod 126a can include an aperture 127 (or recess 127). The aperture 127 can be defined by a portion of the second reciprocation rod 126a. The aperture 127 can extend entirely through the second reciprocation rod 126a. The aperture 127 can be accessed on opposing sides of the second reciprocation rod 126a. In other examples of embodiments, the aperture 127 can include recesses 127 positioned on opposing sides of the second reciprocation rod 126a.

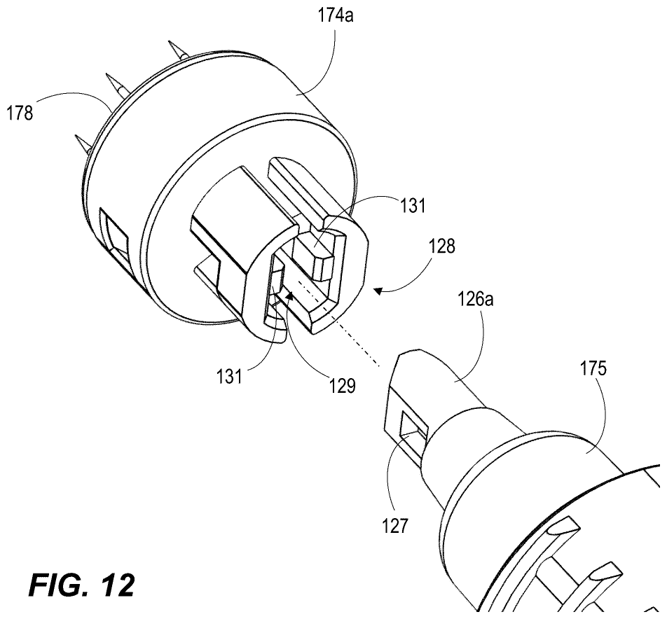

With reference specifically to FIG. 12, the cup 174a can include a fastener assembly 128. The fastener assembly 128 is positioned on the cup 174a on a side opposite the needle holder 178. The fastener assembly 128 is configured to mate (or otherwise engage) with the second reciprocation rod 126a. Stated another way, the fastener assembly 128 is complimentary with the second reciprocation rod 126a to facilitate the removable connection between the cup 174a and the second reciprocation rod 126a.

The fastener assembly 128 includes a channel 129 that is configured to receive an end of the second reciprocation rod 126a. The fastener assembly 128 can also include at least one projection 131. The at least one projection 131 is configured to be received by the aperture 127 (or recess 127) defined by the second reciprocation rod 126a. In the illustrated embodiment, the fastener assembly 128 includes a plurality of projections 131. For example, the fastener assembly 128 includes a pair of projections 131. Each projection 131 is configured to be received by an associated aperture 127 (or recess 127) to selectively fasten the cup 174a to the second reciprocation rod 126a.

With reference back to FIGS. 9-12, a bellows 175 (or a guard member 175) is positioned on the second reciprocation rod 126a. The second reciprocation rod 126a is received by a central aperture defined by the bellows 175. Thus, the second reciprocation rod 126a is configured to slide (or reciprocate) relative to the bellows 175. The bellows 175 is positioned to shield ingress of fluids, liquids, or other undesirable debris into the first cartridge housing 94 (or into the intermediate housing 118 (shown in FIG. 9)).

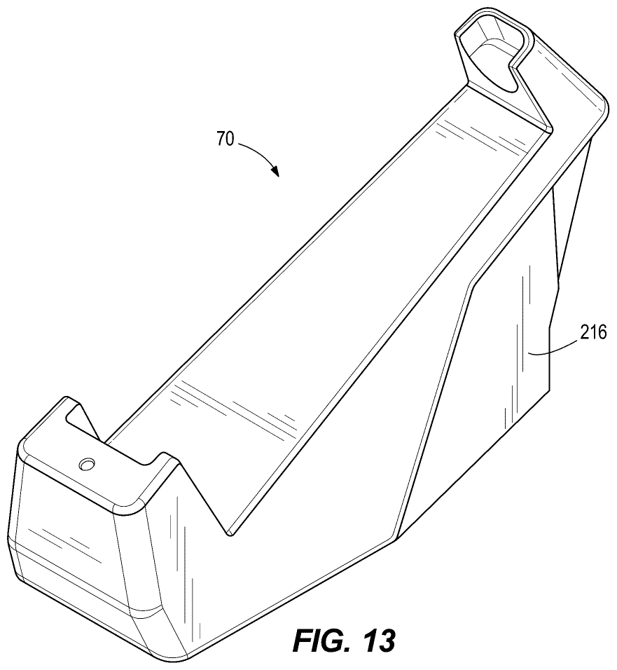

FIG. 13 illustrates the charger 70 configured to charge the battery 66 of the pen 14. The charger 70 includes a charger housing 216 and a plurality of electrical components within the charger housing 216. In some embodiments, the charger 70 can be connected to an external power source by a power cord. In these embodiments, the charger 70 transfers power from the external power source to the battery 66. In other embodiments, the charger 70 can include a power source within the charger housing 216. The power source can be, for example, a battery. In these embodiments, the charger 70 transfer power from the battery inside the charger housing 216 to the battery 66.

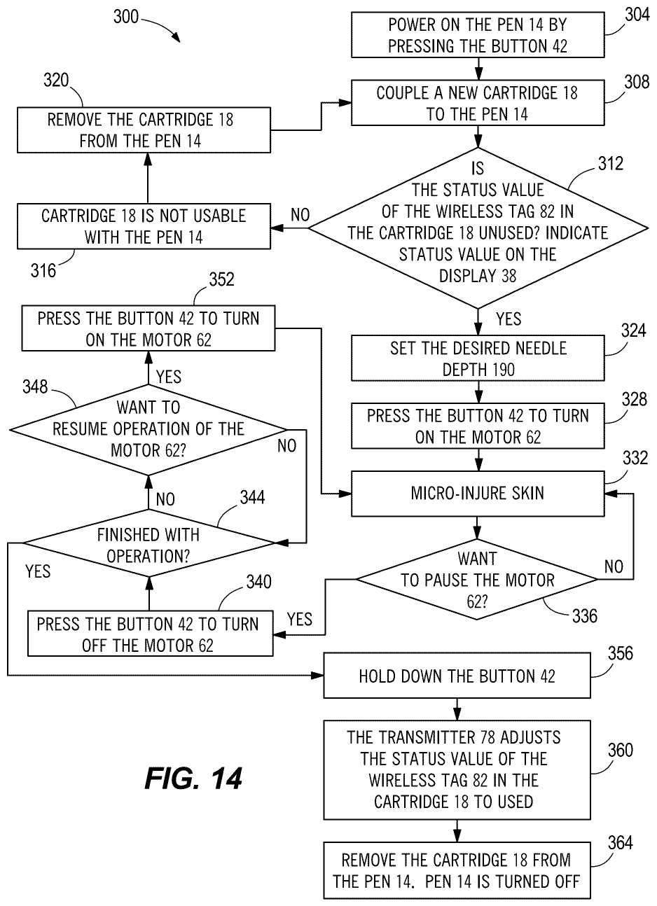

With reference to FIG. 14, an example of a method or process of operating the collagen stimulation assembly 300 is illustrated. The process 300 utilizes the collagen stimulation assembly 10. The process 300 includes a plurality of instructions or steps that are depicted in flow diagram form. The process 300 can be conducted by an operator on a patient. The operator can be a licensed medical professional, or anyone qualified to operate the collagen stimulation assembly 10. The patient can be a person having skin upon which the process 300 is conducted. In some situations, the operator and the patient may the same person.

The process 300 begins at step 304, where the power of the pen 14 is turned on. In some examples of embodiments, the pen 14 can be turned on by pressing the button 42. Once the pen 14 is turned on, the display 38 will be illuminated.

At step 308, an operator couples one cartridge 18 to the pen 14. Specifically, the attachment shaft 50 is inserted into the receiving bore 110. The cartridge 18 is then rotated relative to the pen 14 such that the first indicia 54 on the pen 14 is aligned with the lock symbol of the indicia 58 on the cartridge 18. The cartridge 18 and/or pen 14 can output tactile feedback to further indicate that the cartridge 18 is locked to the pen 14.

At step 312, the transmitter 78 determines whether the cartridge 18 is unused. The transmitter 78 detects the status of the wireless tag 82 in the cartridge 18. The status is either the first value (also referred to as unused) or the second value (also referred to as used). The status of the wireless tag 82 can be displayed for the operator on the display 38. If the status is used, the cartridge 18 has already been used with the pen 14 (or a different pen). If the status is unused, the cartridge 18 has not been used with the pen 14 (or a different pen). The transmitter 78 can also determine if the cartridge 18 is valid. Stated another way, the transmitter 78 determines if the cartridge 18 is compatible with the pen 14. Cartridges of a different type or model may not be compatible with the pen 14. If the cartridge 18 is invalid and/or used, the process 300 moves to step 314.

At step 316, the cartridge 18 is not operable with the pen 14. Any actuation of the button 42 will not result in rotation of the motor 62 and thus reciprocation of the needles 22. For example, the motor 62 may be prevented from operating by the controller 74.

At step 320, an operator removes the cartridge 18 from the pen 14. The cartridge 18 is rotated relative to the pen 14 such that the first indicia 54 on the pen 14 is aligned with the unlock symbol of the indicia on the cartridge 18. The cartridge 18 and/or pen 14 can output tactile feedback to further indicate that the cartridge 18 is unlocked from the pen 14. A new cartridge 18 can be acquired. Once a new (or different) cartridge 18 is acquired, the process 300 returns to step 308.

Returning back to step 312, if yes, the transmitter 78 detects that the cartridge 18 is valid and unused, and the process 300 proceeds to step 324. At step 324, the needle depth 190 can be adjusted. The needle depth 190 is adjusted by rotating the second cartridge housing 98 relative to the intermediate housing 118 about the first and second threads 194, 198. The second cartridge housing 98 is rotated until the pointer 206 is aligned with the desired value of the measurement indicia 202, which indicates that the desired needle depth 190 is achieved. The cartridge 18 and/or pen 14 can output tactile feedback to further indicate that the desired needle depth 190 is achieved.

Next at step 328, the motor 62 is activated. In some examples of embodiments, the motor 62 can be activated by pressing the button 42. Once the motor 62 is activated, the motor 62 receives power from the battery 66 and rotates the attachment shaft 50 about the axis 112. Rotation of the attachment shaft 50 rotates the intermediate drive 106 therewith. Rotation of the intermediate drive 106 about the axis 112 causes the slider 166 to move along the channel 162. As the slider 166 moves along the channel 162, the first reciprocation rod 122, the second reciprocation rod 126, and the needles 22 are reciprocated along the axis 112. Accordingly, in response to activation of the motor 62, the plurality of needles 22 reciprocate along the axis 112.

At step 332, the operator presses the operational end 90 of the cartridge 18 against the skin of the patient. The reciprocating needles 22 can extend through the epidermis of the skin and into the dermis, which causes injury to the skin. This injury to the skin can stimulate controlled growth of collagen in the skin. Collagen growth in the skin can restore elasticity to older skin, reduce appearances of scars, and/or improve the overall appearance and/or structure of the skin. The operator can continue to micro-injure the skin to stimulate collagen growth as needed.

During operation of the collagen stimulation assembly 10 and associated micro-injury to the skin of the patient to simulate collagen growth, the operator may want to pause operation of the assembly 10, and associated reciprocation of the needles 22. A pause in operation is associated with a delay or break in operation with the intent to return to operation with the patient. If no, no pause is needed, the operator continues with step 332 and treatment by micro-injuring the skin of the patient. If yes, a pause is needed, the process 300 proceeds to step 340.

At step 340, the motor 62 is turned off. In some examples of embodiments, the motor 62 can be turned off by pressing the button 42. Once power from the motor 62 is shut off, the needles 22 stop reciprocating.

At step 344, the collagen stimulation assembly 10 is paused and the needles 22 are not reciprocating. At this step, the operator determines whether the operation is finished or if reciprocation of the needles 22 will resume. If no, the operator plans to continue reciprocating the needles 22.

At step 348, the operator decides whether to turn the motor 62 back on to continue micro-injuring the patient's skin. If not, the process 300 returns to step 344. During step 344 and/or step 348, the operator can adjust the needle depth 190. For example, the operator may want to increase the needle depth 190. Additionally, during step 344 and/or step 348, the operator and patient may be taking a break, the operator may be analyzing the skin of the patient, the operator may be preparing to micro-injure a different area of the patient's skin, etc. If yes, the process proceeds to step 352.

At step 352, the motor 62 is turned on. In some examples of embodiments, the motor 62 is turned on by pressing the button 42. Step 352 is the same as step 328. As such, step 352 can include any features as described in reference to step 328. After step 352, the process 300 returns to step 332.

Returning to step 344, if yes, the operator is done micro-injuring the skin of the patient. For example, the operator may have completed the desired treatment on the patient. The process 300 proceeds to step 356. At step 356, the motor 62 is paused and the operator initiates a power down of the motor 62. In some examples of embodiments, the power down of the motor 62 is initiated by holding down the button 42. The operator can hold down the button 42 for a specified time period (e.g., two seconds, three seconds, etc.). Once the button 42 has been held down for the specified period of time, the process proceeds to step 360.

At step 360, the transmitter 78 adjusts the status of the wireless tag 82 on the cartridge 18 to used or the second value. This will prevent the cartridge 18 from being used again with the pen 14 (or a different pen).

At step 364, the display 38 may signal to the operator that the cartridge 18 can be removed from the pen 14. The operator then rotates the cartridge 18 relative to the pen 14 such that the first indicia 54 on the pen 14 is aligned with the unlock symbol of the indicia on the cartridge 18. The cartridge 18 and/or pen 14 can output tactile feedback to further indicate that the cartridge 18 is unlocked from the pen 14. The cartridge 18 is then removed from the pen 14. Once the cartridge 18 is removed from the pen 14, the pen 14 turns off. In some embodiments, the pen 14 may turn off a set amount of time after the cartridge 18 is removed from the pen 14 (e.g., three seconds, five seconds, etc.).

In some embodiments, step 360 can be positioned after step 364. In these embodiments, the transmitter 78 adjusts the status of the wireless tag 82 immediately after the cartridge 18 is removed from the pen 14. For example, rotation of the cartridge 18 relative to the pen 14 can prompt the transmitter 78 to adjust the status of the wireless tag 82 to the pen 14. It should be appreciated that the process 300 can include any suitable additional or replacement steps for properly ensuring that an old or used cartridge 18 is not used again.

Although the invention has been described in detail with reference to certain preferred embodiments, variations and modifications exist within the scope and spirit of one or more independent aspects of the invention as described. Various features and advantages of the invention are set forth in the following claims.

What is claimed is:

1. A collagen stimulation assembly comprising:
a pen including
a motor,
a first attachment portion configured to be rotated by the motor about an axis, and
a transmitter; and
a single use cartridge including
a second attachment portion coupled to the first attachment portion, the second attachment portion configured to be rotated about the axis by the first attachment portion,
a reciprocation assembly connected to the second attachment portion, the reciprocation assembly configured to move along the axis,
a plurality of needles connected to the reciprocation assembly, the plurality of needles configured to move along the axis with the reciprocation assembly, and
an RFID tag configured to interact with the transmitter;
wherein the transmitter detects a status of the RFID tag in response to the second attachment portion coupling to the first attachment portion,
wherein the motor can operate when the status is a first value,
wherein the motor is prevented from operating when the status is a second value, and
wherein the transmitter changes the status to the second value in response to the second attachment portion detaching from the first attachment portion.

2. The collagen stimulation assembly of claim 1, wherein the status of the RFID tag is the first value when the single use cartridge has not been used before.

3. The collagen stimulation assembly of claim 2, wherein the status of the RFID tag is the second value when the single use cartridge has been used before.

4. The collagen stimulation assembly of claim 1, wherein the pen further comprises a button configured to adjust operation of the motor.

5. The collagen stimulation assembly of claim 1, wherein the pen further comprises a display configured to indicate the status of the RFID tag in response to the second attachment portion coupling to the first attachment portion.

6. The collagen stimulation assembly of claim 1, wherein the first attachment portion has a star shape, and wherein the second attachment portion has the star shape.

7. The collagen stimulation assembly of claim 1, wherein the transmitter is positioned adjacent the first attachment portion, and wherein the RFID tag is positioned adjacent the second attachment portion.

8. The collagen stimulation assembly of claim 1, wherein the single use cartridge is a first of a plurality of single use cartridges, and wherein the second attachment portion of each single use cartridge is configured to be coupled to the first attachment portion of the pen.

9. A collagen stimulation assembly comprising:
a pen including
a motor,
a first star shaped attachment portion configured to be rotated by the motor about an axis, and
a transmitter; and
a single use cartridge including
a second star shaped attachment portion coupled to the first star shaped attachment portion, the second star shaped attachment portion configured to be rotated about the axis by the first star shaped attachment portion,
a reciprocation assembly connected to the second star shaped attachment portion, the reciprocation assembly configured to move along the axis,
a plurality of needles connected to the reciprocation assembly, the plurality of needles configured to move along the axis with the reciprocation assembly, and
a wireless tag configured to interact with the transmitter;
wherein the transmitter detects a status of the wireless tag in response to the second attachment portion coupling to the first attachment portion,
wherein the motor can operate when the status is a first value,
wherein the motor is prevented from operating when the status is a second value, and
wherein the transmitter changes the status to the second value in response to the second attachment portion detaching from the first attachment portion.

10. The collagen stimulation assembly of claim 9, wherein the status of the wireless tag is the first value when the single use cartridge has not been used before.

11. The collagen stimulation assembly of claim 10, wherein the status of the wireless tag is the second value when the single use cartridge has been used before.

12. The collagen stimulation assembly of claim 9, wherein the pen further comprises a button configured to adjust operation of the motor.

13. The collagen stimulation assembly of claim 9, wherein the pen further comprises a display configured to indicate the status of the wireless tag in response to the second attachment portion coupling to the first attachment portion.

14. The collagen stimulation assembly of claim 9, wherein the transmitter is positioned adjacent the first attachment portion, and wherein the wireless tag is positioned adjacent the second attachment portion.

15. The collagen stimulation assembly of claim 9, wherein the wireless tag is an RFID tag.

16. A method of operating a collagen stimulation assembly including a pen and a single use cartridge, the pen having a motor, a first attachment portion configured to be rotated by the motor about an axis, and a transmitter, the single use cartridge having a second attachment portion, a reciprocation assembly connected to the second attachment portion, a plurality of needles connected to the reciprocation assembly, and a wireless tag, the method comprising:
connecting the second attachment portion to the first connection portion;
detecting a status of the wireless tag with the transmitter, the status being a first value or a second value;
preventing operation of the motor in response to the status being the second value;
operating the motor in response to the status being the first value, operating the motor causing the first attachment portion and the second attachment portion to rotate about the axis, rotation of the second attachment portion causing the reciprocation assembly and the plurality of needles to reciprocate along the axis;
ending operation of the motor;
detaching the second attachment portion from the first connection portion; and
adjusting the status of the wireless tag to the second value with the transmitter.

17. The method of claim 16, further comprising pausing operation of the motor by pressing a button on the pen and resuming operation of the motor by pressing the button.

18. The method of claim 16, wherein the status of the wireless tag is the first value when the single use cartridge has not been used before, and wherein the status of the wireless tag is the second value when the single use cartridge has been used before.

19. The method of claim 16, wherein the wireless tag is an RFID tag.

20. The method of claim 16, wherein the first attachment portion has a star shape, and wherein the second attachment portion has the star shape.
